(19) United States
(12) Patent Application Publication
(10) Pub. No.: US 2006/0134617 A1
(43) Pub. Date: Jun. 22, 2006

(54) Enzymatic method for the isolation of dna from plant tissue

(75) Inventors: Jean-Francois Manen, Arculinge (FR); Arkady Sinitsyn, Moscow (RU); Olga Arkadievna Sinitsyna, Moscow (RU)

Correspondence Address: SONNENSCHEIN NATH & ROSENTHAL LLP;FOR PAULA EVANS, P.O. BOX 061080, WACKER DRIVE STATION, SEARS TOWER, CHICAGO, IL, 60606-1080, US

(73) Assignee: University of Geneva

(21) Appl. No.: 10/534,801

(22) PCT Filed: Dec. 18, 2003
(86) PCT No.: PCT/IB03/06422

§ 371 Date: Dec. 19, 2005

Related U.S. Application Data
(60) Provisional application No. 60/434,699, filed on Dec. 18, 2002.

(51) Int. Cl. C12Q 1/68; C12N 9/24; C12N 1/08; C12N 1/16

(52) U.S. Cl. 435/6; 435/200; 435/270; 435/254.3

(57) ABSTRACT

The invention relates to a mixture of cell wall degrading enzymes. The invention further relates to methods for isolating DNA from plant tissue that utilize a mixture of cell wall degrading enzymes. The invention also relates to kits for isolating DNA from plant tissue wherein the kit comprises a mixture of cell wall degrading enzymes.

### FIELD OF THE INVENTION

The invention relates to a mixture of cell wall degrading enzymes, and methods of using this mixture to isolate DNA from plant tissue

### BACKGROUND

Automated, high-throughput systems for the isolation of genomic DNA are required when the DNA of hundreds or thousands of organisms are to be analyzed and compared, for example for population genetics, species identification, biodiversity investigation, selection screening or pathogen screening. Such large scale automated DNA mini-prep facilities are recently offered by several companies for animal tissues. However, plant tissues, because of their cell walls, are recalcitrant to the automation of DNA isolation.

The release of DNA from plant tissue requires the disruption of the cell wall. The distinguishing feature of plant cells is the presence of a cell wall, a complex mixture of carbohydrate polymers and proteins. The cell wall is classified as primary and secondary cell wall depending on the growth stage of its deposition. Its composition can vary from species to species, and is different in dicots and in monocots (Reiter, 1998, Trends Plant Science, 3:27-32). Excluding several classes of proteins and glycoproteins, the major components of the cell wall are cellulose, hemicellulose, pectic polysaccharides (in the middle lamella and cell walls) and lignin (in the secondary wall). The digestion of the cell wall is well documented for the isolation of protoplasts (Evans et al., 1977, Isolated Plant Protoplasts, In “Plant tissue and Cell Culture”, pp. 61-136, H. E. Street ed., Blackwell Scientific Publications, Oxford).

The methods of DNA isolation from plant tissue that are known in the art generally involve physical grinding of the tissue followed by extraction procedures in various buffers containing detergents and EDTA. Because of the necessary initial grinding step, these methods are time consuming, not reproducible, and not compatible with automation.

Disruption of the cell wall is typically done mechanically in a mortar with a pestle, in a microtube with a piston or in a rotating or shaking mill in the presence of abrasive beads. Qiagen has developed a system (DNeasy_{—}96_plant) in which the grinding of leaves is performed in 96 or 192-well plates installed in a mixer mill in the presence of tungsten carbide beads (world wide web at qiagen.com/catalog/). Although the method yields amounts of DNA that would be sufficient for most applications, it necessarily requires a centrifugation step to remove cell debris and can therefore not be fully automated. Moreover, the grinding step can cause DNA shearing, and the resulting low-molecular weight DNA is not suitable for many applications, including but not limited to AFLP (Amplified Fragment Length Polymorphism), RAPD (Random Amplified Polymorphic DNA), RFLP (Restriction Fragment Lenght Polymorphism), Southern blotting and partial mini-genomic libraries

Two methods have been designed to avoid the mechanical disruption of plant tissues necessary for the isolation of their DNA. In 1993, a method was developed which is based on the disruption of plant cell walls using xanthate-forming compounds (U.S. Pat. No. 5,204,246). A kit for easy extraction of plant PCR ready DNA is now commercially available from Sigma-Aldrich (Extract-N-amp Plant PCR kit, world wide web at sigma-aldrich.com/saws.nsf/SigProducts). The method of this kit involves the incubation of a 0.5 cm leaf disk in an extraction solution for 10 min. at 95° C. and direct PCR of the supernatant diluted in a Dilution Solution. This method was originally developed for the detection of Cannabis, but is predicted to work for other species. Both methods produce low amounts of DNA, typically less than 10% of the amount of DNA produced with the grinding method. Therefore, although the quality of the DNA is generally good, DNA isolated by these two methods can only be used for PCR.

There is a need in the art for a method of isolating high yields of DNA from plant tissues that does not require a physical grinding step.

There is also a need in the art for a method of isolating high yields of high molecular weight DNA from plant tissues.

There is also a need in the art for an automated, high throughput system for the isolation of plant DNA.

There is also a need in the art for a method of isolating DNA from plant tissues wherein the DNA can be used for a variety of applications including but not limited to PCR, sequencing, cloning, cell transduction, AFLP, RAPD, RFLP, SSRP, Southern blotting, partial mini-genomic libraries. etc . . .

### SUMMARY

The invention relates to a mixture of cell wall degrading enzymes. The invention provides for compositions and kits comprising this enzyme mixture. The invention also provides for a method of isolating DNA from plant tissue wherein plant tissue is incubated with a mixture of cell wall degrading enzymes.

The method of the invention can be used in a high throughput system that can easily be automated. Numerous samples can be processed simultaneously using the method and compositions of the invention. The method of the invention allows for the isolation of high yields of DNA from plant tissue in the absence of a mechanical step for breaking apart or grinding the plant tissues. The DNA isolation method of the invention does not require a centrifugation step or hazardous chemicals. The DNA that is isolated according to this method can be used for a wide variety of applications, including PCR, AFLP, RAPD, RFLP, SSRP, Southern blotting and partial mini-genomic libraries.

The invention provides for a composition comprising a mixture of cell wall degrading enzymes.

In one embodiment, the enzymes of the composition are produced recombinantly.

In another embodiment, the mixture of enzymes is isolated from a microorganism.

As used herein, the term “isolated” relates to the isolation of a mixture of enzymes that comprises more than 50% (by weight), for example 51, 55, 60, 65, 70, 75, 80, 85, 90, 95, 99, 100% or more contaminating polypeptides, polynucleotides, or contaminating material derived from the microorganism of an unlike nature from the purified components of the mixture (e.g., with less than 95%-100% sequence identity). For example, an isolated mixture of cell wall degrading enzymes includes an ultrafiltrate concentrate of a microorganism, as described herein.

“Isolated” also includes “purified. As used herein, “purified” and like terms relate to the isolation of a mixture of enzymes in a form that is substantially free of contaminants normally associated with the enzymes in a native or natural environment. For example, a “purified” mixture of cell wall degrading enzymes preferably comprises less than 50% (by weight), preferably less than 40%, and most preferably, less than 2% contaminating polypeptides, polynucleotides, or contaminating material derived from the microorganism of an unlike nature from the purified components of the mixture (e.g., with less than 95%-100% sequence identity).

In another embodiment, the microorganism is a fingus for example, from the group consisting of: Trichoderma, Pencillium and Aspergillus. In another embodiment, the fungus is the TW-1 mutant strain of Trichoderna longibrachiatum (deposited in Russian Collection of Microorganisms, deposition number VKMF-3634D).

In another embodiment, the composition comprises a mixture of cell wall degrading enzymes comprising carbohydrases. In another embodiment, the mixture comprises cellulases, β-glucanases, mannanases, xyloglucanases, pectinases, glycosidases and xylanases. The invention also contemplates a composition that is a mixture of enzymes comprising at least one of the following enzymes: cellulases, β-glucanases, mannanases, xyloglucanases, pectinases, glycosidases and xylanases.

In another embodiment, the composition further comprises a digestion buffer comprising a DNA preserving agent, as defined herein.

As used herein, “digestion buffer” refers to a buffer that supports the activity of the mixture of cell wall degrading enzymes, such that the enzyme mixture decomposes or breaks down a plant tissue such that DNA can be isolated from the tissue using the mixture of cell wall degrading enzymes of the invention. A “digestion buffer” preferably comprises at least one of a DNA preserving agent, as described herein, a non-ionic detergent, polyethylene glycol (PEG), a buffering agent and salt. In another embodiment, one ml of a digestion buffer consists of 300 μl of 0.5 M EDTA, pH 8.0, 350 μl of 0.3 M sodium acetate adjusted to pH 4.6 with acetic acid, 10 μl of Triton X100, 10 μl of 25% PEG 8000 (Sigma P-5413) and 330 μl of water, and has a final pH of 5 to 6.

As used herein, “DNA preserving agent” refers to an agent, for example, EDTA, that decreases or prevents degradation of DNA. Preferably, in the presence of a DNA preserving agent of the invention, DNA degradation is <15%, more preferably <10% and most preferably, <5% of the total amount of DNA initially present in a sample or as compared to a replicate sample that is incubated in the absence of a DNA preserving agent.

In another embodiment, the digestion buffer comprises EDTA.

In another embodiment, the digestion buffer comprises EDTA and at least one of a non-ionic detergent and PEG.

In another embodiment, the detergent is Triton-X-100. A “detergent” according to the invention also includes any non-ionic detergent, for example, NP-40 or Tween-20.

In another embodiment, the digestion buffer has a pH of 5.0.

The invention also provides for a method for isolating DNA from plant tissue comprising combining a sample of plant tissue with a composition comprising a mixture of cell wall degrading enzymes, and incubating them under certain conditions.

As used herein, “combining” refers to mixing, in any order, a sample of plant tissue and a mixture of cell wall degrading enzymes.

In another embodiment, the enzymes of the mixture are prepared recombinantly.

In another embodiment, the mixture of cell wall degrading enzymes of the method is isolated from a microorganism. The microorganism is a fungus, for example, selected from the group consisting of: Trichoderma, Pencilliun and Aspergillus and the TW-1 mutant strain of Trichoderma longibrachiatum (deposited in Russian Collection of Microorganisms, deposition number VKMF-3634D).

In another embodiment, the mixture of cell wall degrading enzymes of the method comprises carbohydrases. In another embodiment, the mixture of cell wall degrading enzymes of the method comprises cellulases, β-glucanases, mannanases, xyloglucanases, pectinases, glycosidases and xylanases. In another embodiment, the mixture of cell wall degrading enzymes of the method comprises at least one of of the following enzymes: cellulases, β-glucanases, mannanases, xyloglucanases, pectinases, glycosidases and xylanases.

In another embodiment, the composition used in the method further comprises a digestion buffer comprising a DNA preserving agent.

In another embodiment, the DNA preserving agent is EDTA.

In another embodiment, the digestion buffer comprises a DNA preserving agent and at least one of a non-ionic detergent and PEG.

In another embodiment, the detergent is Triton-X-100.

In another embodiment, the digestion buffer has a pH of 5.0.

In another embodiment, the incubation step is performed at 50° C.

In another embodiment, the mixture of cell wall degrading enzymes and the sample are agitated at 250 rpm for 1-16 hours. For example, the mixture is agitated for 1, 2, 3, 4, 5, 6, 7, 8, 9, 10, 11, 12, 13, 14, 15 or 16 hours.

In another embodiment, the method further comprises the steps of adding a DNA-binding solid support and binding the DNA to the solid support after the incubation step.

As used herein, a “solid support” refers to a substrate that is capable of forming non-covalent linkages with a molecule, for example nucleic acid. The support can be either biological in nature, such as, without limitation, a cell or bacteriophage particle, or synthetic, such as, without limitation, an acrylamide derivative, agarose, cellulose, nylon, silica, or paramagnetic particles. In one embodiment, a solid surface comprises functionalized silica or agarose beads. In another embodiment, the solid support is paramagnetic beads. A “solid support” also refers to a solid object or surface upon which a nucleic acid sample is deposited. Solid supports include, but are not limited to, glass, metals, polyacrylamide, polystyrene, polypropylene, polyethylene, dextran, nylon, ceramics, silica and silicon. Solid supports can comprise flat (planar) surfaces or non-planar surfaces, such as the surfaces of microbeads.

In another embodiment, the method is automated.

As used herein, “automated” refers to operated automatically by mechanical or electronic devices, for example robots, well known in the art.

The invention also provides for a kit for isolating DNA from plant tissue comprising a mixture of cell wall degrading enzymes and packaging means thereof.

In one embodiment, the enzymes of the mixture are prepared recombinantly.

In one embodiment, the mixture of cell wall degrading enzymes of the kit is isolated from a microorganism. The microorganism is a fungus, for example, selected from the group consisting of: Trichodenna, Pencillium and Aspergillus and the TW-1 mutant strain of Trichoderma longibrachiatum (deposited in Russian Collection of Microorganisms, deposition number VKMF-3634D).

In another embodiment, the mixture of cell wall degrading enzymes of the kit comprises carbohydrases. In another embodiment, the enzyme mixture of the kit comprises cellulases, β-glucanases, mannanases, xyloglucanases, pectinases, glycosidases and xylanases. In another embodiment, the enzyme mixture of the kit comprises at least one of the following enzymes: cellulases, β-glucanases, mannanases, xyloglucanases, pectinases, glycosidases and xylanases.

In another embodiment, the kit further comprises a digestion buffer comprising a DNA preserving agent.

In another embodiment, the DNA preserving agent is EDTA.

In another embodiment, the digestion buffer comprises a DNA preserving agent and at least one of a non-ionic detergent and PEG.

In another embodiment, the detergent is Triton-X-100.

In another embodiment, the digestion buffer has a pH of 5.0.

In another embodiment, the kit further comprises a DNA-binding solid support.

In another embodiment, the kit further comprises buffers for DNA isolation with a solid support.

Further features and advantages of the invention will become more fully apparent in the following description of the embodiments and drawings thereof, and from the claims.

### BRIEF DESCRIPTION OF THE DRAWINGS

FIG. 1 is a 96 well plate wherein certain wells of the plate contain a piece of a plant leaf, and a mixture of cell wall degrading enzymes.

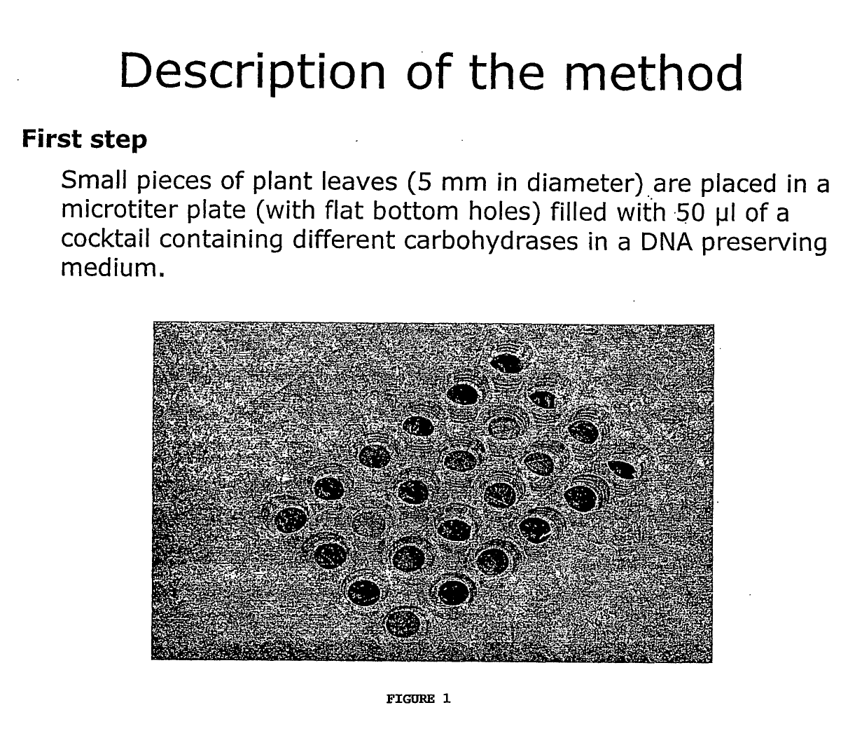

FIG. 2 depicts plant leaves (Acer, Betula and Ilex) before (A, B, C) and after (D, E, F) digestion with a mixture of cell wall degrading enzymes of the invention.

FIG. 3 is an agarose gel depicting DNA isolated from silica gel-dried leaves of Ilex perado (A), PCR amplification of atpB-rbcL (plastid DNA) (B) and PCR amplification of ITS (nuclear DNA) wherein the DNA was isolated using a mixture of cell wall degrading enzymes of the invention.

FIG. 4 is an agarose gel demonstrating DNA isolated from 24 different plant species using the mixture of cell wall degrading enzymes of the invention.

### DETAILED DESCRIPTION

The invention relates to a mixture of cell wall degrading enzymes that have been isolated from a microorganism or prepared by recombinant methods. This enzyme mixture is used to isolate DNA from plant tissues, in the absence of a mechanical disruption step. This method can be easily adapted for high throughput, automated applications.

Enzyme Mixture

The enzyme mixture of the invention comprises a cocktail of extracellular secreted enzymes purified from a microorganism or prepared via recombinant methods.

Isolation of a Mixture of Cell Wall Degrading Enzymes from a Microorganism

A mixture of cell wall degrading enzymes is isolated from a microorganism by cultivating the microorganism in a fermenter (for example for 144 hours), removing fingal cell. biomass by filtration, centrifugation or an appropriate method of separation known in the art, and concentrating the cultural filtrate by ultra filtration. The resulting ultrafiltrate concentrate can be used directly for the isolation of DNA from plant tissue.

Mixtures of cell wall degrading enzymes useful according to the invention are isolated from soil living microorganisms, particularly fuingi. Among fingal strains, Trichoiderma, Penicillium, and Aspergillus represent the major inhabitants of soil. In particular, mixtures of cell wall degrading enzymes of the invention are purified from different species of Trichoiderma, Penicillium and Aspergillus.

An enzyme mixture useful according to the invention is isolated from a mutated strain of Trichoderma, particularly Trichoderma longibrachiatum Rifai TW-1 (syn Trichoderma reesi Simmons), deposited in Russian Collection of Microorganisms, deposition number VKMF-3634D. Strains of Trichoderma longibrachiatum Rifai TW-1 VKMF-3634D produce a mixture of the following carbohydrases causing the effective decomposition (degradation, hydrolysis) of polysaccharides of a plant cell wall: cellulases, β-glucanases, xylanase, mannanases, xyloglucanases, pectinases, glycosidases (such as β-glucosidase, β-xylosidase, α-L-arabinofuranosidase, α-galactosidase).

Preferably, the mixture of cell wall degrading enzymes is produced from the strain Trichoderma longibrachiatum Rifai TW-1 VKMF-3634D that is cultivated on a specially selected fermentation media, consisting of wheat bran (20-30 g/L), corn step solid (20-30 g/L), hydrolyzed starch (40-50 g/L), mineral salts and with feeding by lactose (feeding by 25% solution of lactose with feed rate 50 mL per hour, start feeding after 48 hours beginning of fermentation).

The ratio of activities of the mixture of cell wall degrading enzymes produced by strain Trichoderma longibrachiatum Rifai TW-1 VKMF-3634D is the following: cellulase-1-2, β-glucanase-1-2, xylanase-0.3-0.6, mannanase-0.02-0.04, xyloglucanases-0.1-0.2, pectinases-0.06-0.12, β-glucosidase-0.01-0.02, β-xylosidase-0.002-0.004, α-L-arabinofuranosidase-0.01-0.02, α-galactosidase-0.002-0.004).

Activities of the enzymes present in a mixture of cell wall degrading enzymes purified from a microorganism, for example from the strain Trichodenna longibrachiatuin Rifai TW-1 VKMF-3634D are in the following range (in liquid ultrafiltration-concentrated form of preparation): cellulase-250-50,000 and preferably 2500-5000 of U/ml, β-glucanase-240-48,000 and preferably 2400-4800 U/mL, xylanase-40-18,000 and preferably, 400-1800 U/mL, mannanase-5-1000 and preferably 50-100 U/mL, xyloglucanases-25-5000 and preferably 250-500 U/mL, pectinases-15-3000, preferably, 150-300 U/mL, β-glucosidase-2.5-500, preferably 25-50 U/mL, β-xylosidase-0.5-100, preferably 5-10 U/mL, α-L-arabinofuranosidase-2.5-500, preferably 25-50 U/mL, α-galactosidase-0.5-100, preferably, 5-10 U/mL.

One Unit of CMCase, β-glucanase, xylanase, mannanase, xyloglucanase and pectinase activities is equal to the amount of enzyme which liberates 1 micromole of reducing sugars expressed as glucose equivalents in one minute when reaction of hydrolysis of these substrates is carried out during 10 min at 50° C. and pH 5.0 (0.05 M sodium acetate buffer) and reducing sugars are measured by Somogyi-Nelson assay.

One unit of β-glucosidase, β-xylosidase, α-L-arabinofuranosidase and α-galactosidase activities is equal to the amount of enzyme which liberates 1 micromole of pNP in one minute when a hydrolysis reaction of these substrates is carried out for 10 min at 40° C. and pH 5.0 (0.05 M sodium acetate buffer) and pNP is measured at 400 nm.

Cellulase activity is assayed by determining the initial velocity of reducing sugar production from carboxymethylcellulose (CMC) at 50° C. and pH 5.0. β-glucanase activity is assayed by determining the initial velocity of reducing sugar production from barley β-glucan at 50° C. and pH 5.0. Xylanase activity is determined by assaying the initial velocity of reducing sugar production from wheat arabinoxylan at 50° C. and pH 5.0. Mannanase activity is determined by assaying the initial velocity of reducing sugar production from Gleditcia treacanthos galactomannan at 50° C. and pH 5.0. Xyloglucanase activity is determined by assaying the initial velocity of reducing sugar production from tamarind seed xyloglucan at 50° C. and pH 5.0. Pectinase activity is determined by assaying the initial velocity of reducing sugar production from K-salt of polygalacturonic acid at 50° C. and pH 5.0. β-glucosidase activity is assayed by determining the initial velocity of p-nitrophenol (pNP) release from pNP-β-glucoside at 40° C. and pH 5.0. β-xylosidase activity is assayed by measuring pNP release from pNP-β-xyloside at 40° C. and pH 5.0. α-L-arabinofuranosidase activity is assayed by detecting pNP release from pNP-α-L-arabinofuranoside at 40° C. and pH 5.0. α-galactosidase activity is assayed by measuring pNP release from pNP-α-galactoside at 40° C. and pH 5.0.

Preparation of a Mixture of Cell Wall Degrading Enzymes Via Recombinant Methods

Enzymes useful according to the invention are prepared by recombinant methods known in the art and described herein. Cell wall degrading enzymes are prepared via recombinant technology, isolated and combined to prepare the mixture of the invention. In one embodiment, enzymes are combined in the ratios described above.

Expression vectors for preparing recombinant forms of the enzymes of the invention can be prepared according to methods known in the art from the following sequences.

Endo-1,4-β-glucanase (“cellulase”), EC 3.2.1.4

Q12714, EGL1, Trichoderma longibrachiatum
P07981, EGL1, Trichoderma reesei
   Endo-1,3(1,4)-β-glucanase (“β-glucanase”), EC 3.2.1.6
Q8GRB4, BG1314, Bacillus licheniformis
O60019, BG1, Phaffia rhodozyma
Q59328, LICA, Clostridium thermocellum
   Endo-xylanase (“xylanase”), EC 3.2.18
Q02244, XLN2, Trichoderma reesei
P55328, XYNA, Aspergillus awanori
   β-mannanase, EC 3.2.1.78
P51529, MANA, Streptomyces lividans
   Polygalacturonase (“pectinase”), EC 3.2.1.15
O59925, PEG1, Penicillium expansum
P26213, PG1, Aspergillus niger
   β-glucosidase, EC 3.2.1.21
P48825, BGL1, Aspergillus awamori
   β-xylosidase, EC 3.2.1.37
P48792, XYL1, Trichoderma koningii
   α-L-arabinofuranosidase, EC 3.2.1.55
Q92455, ABF1, Trichoderma reesei
P42254, ABFA, Aspergillus niger
P42255 ABFB, Aspergillus niger
   α-galactosidase, EC 3.2.1.22
P28351, AGLA, Aspergillus niger
Q9UUZ4, AGLC, Aspergillus niger

Recombinant enzymes of the mixture of the invention are prepared from expression vectors. Expression vectors comprising enzymes of the mixture of the invention are prepared using conventional techniques which are within the skill of the art. Such techniques are described in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

Polynucleotide sequences comprising DNA encoding cell wall degrading enzymes of interest can be isolated from cDNA libraries by cloning methods well known to those skilled in the art (Ausubel et al., supra). The desired clone is preferably identified by hybridization to a nucleic acid probe or by expression of a protein that can be detected by an antibody. Alternatively, the desired clone is identified by polymerase chain amplification of a sequence defined by a particular set of primers according to the methods described below.

Following the identification of a desired cDNA clone containing a particular sequence, polynucleotides of the invention are isolated from these clones by digestion with restriction enzymes, well known to those skilled in the art (Ausubel et al., supra).

Polynucleotide sequences of the invention can be used to express the protein product of the gene of interest by inserting the polynucleotide sequence into an expression vector which contains the necessary elements for the transcription and translation of the inserted coding sequence. Expression vectors suitable for protein expression in mammalian cells, bacterial cells, insect cells or plant cells are well known in the art.

Methods which are well known to those skilled in the art can be used to construct expression vectors containing a protein-encoding sequence and appropriate transcriptional or translational controls. These methods include in vivo recombination or genetic recombination. Such techniques are described in Ausubel et al., supra and Sambrook et al., supra.

A variety of expression vector/host systems may be utilized to contain and express a protein product of a candidate gene according to the invention. These include but are not limited to microorganisms such as bacteria transformed with recombinant bacteriophage, plasmid or cosmid DNA expression vectors; yeast transformed with yeast expression vectors; insect cell systems infected with virus expression vectors (e.g., baculovirus); plant cell systems transfected with virus expression vector (e.g., cauliflower mosaic virus, CaMV; tobacco mosaic virus, TMV) or transformed with bacterial expression vectors (e.g., Ti or pBR322 plasmid); or animal cell systems.

For long-term, high-yield production of recombinant proteins, stable expression is preferred, according to methods known in the art. For example, cell lines which stably express a foreign protein may be transformed using expression vectors which contain viral origins of replication or endogenous expression elements and a selectable marker gene.

The presence of the polynucleotide sequence encoding the protein of interest can be detected by DNA-DNA or DNA-RNA hybridization or amplification using probes, portions or fragments of the sequence encoding the foreign protein of interest (i.e., Southern , northern blotting, PCR, RT-PCR, Q-PCR, RNase protection assays or in situ hybridization.

A variety of protocols for detecting and measuring the expression of the foreign protein, using either polyclonal or monoclonal antibodies specific for the protein are known in the art. Examples include enzyme-linked immunosorbant assay (ELISA), radioimmunoassay (RIA) and fluorescent activated cell sorting (FACS). A two-site, monoclonal-based immunoassay utilizing monoclonal antibodies reactive to two non-interfering epitopes on the protein of interest is preferred, but a competitive binding assay may be employed. These and other assays are described in Hampton et al., 1990, Serological Methods a Laboratory Manual, APS Presds, St Paul Minn. and Maddox., et al., 1983, J Exp Med 158:1211.

Host cells transformed with a nucleotide sequence encoding a protein of interest may be cultured under conditions suitable for the expression and recovery of the encoded protein from cell culture. The protein produced by a recombinant cell may be secreted or contained intracellularly depending on the sequence and/or the vector used. As will be understood by those of skill in the art, expression vectors containing a sequence encoding a protein of interest can be designed with signal sequences which direct secretion of the protein of interest through a prokaryotic or eucaryotic cell membrane. Other recombinant constructions may join the sequence encoding the protein of interest to the nucleotide sequence encoding a polypeptide domain which will facilitate purification of soluble proteins (Kroll et al., 1993, DNA Cell Biol, 12:441).

The protein of interest may also be expressed as a recombinant protein with one or more additional polypeptide domains added to facilitate protein purification. Such purification facilitating domains include, but are not limited to, metal chelating peptides such as a histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor XA or enterokinase (Invitrogen, San Diego Calif.), between the purification domain and the protein of interest is useful for facilitating purification. One such expression vector provides for expression of a fusion protein comprising the sequence encoding a foreign protein and nucleic acid sequence encoding 6 histidine residues followed by thioredoxin and an enterokinase cleavage site. The histidine residues facilitate purification while the enterokinase cleavage site provides a means for purifying the foreign protein from the fusion protein.

Vectors

The invention provides for polynucleotides that can be provided in vectors and used for production of protein of interest.

A vector of the invention contains a nucleic acid of interest under the control of sequences which facilitate the expression of the protein in a particular host cell or cell-free system. The control sequences comprise sequences such as a promoter, and, if necessary enhancers, poly A sites, etc . . . Preferably, the protein is also expressed in such a way as to maintain activity. Preferably, one or more selectable markers are also present on the vector for the maintenance in prokaryotic or eukaryotic cells. Basic cloning vectors are described in Sambrook et al., Molecular Cloning, Molecular Cloning, A Laboratory Manual, Cold Spring Harbor Laboratory Press, (1989). Examples of vectors useful according to the invention include plasmids, bacteriophages, other viral vectors and the like.

Cells

A vector of the invention can be introduced into an appropriate host cell. These cells can be prokaryotic or eukaryotic cells, e.g., bacterial cells, yeast cells, fungi or mammalian cells, and the vector or nucleic acid can be introduced (transformed) into these cells stably or transiently by conventional methods, protocols for which can be found in Sambrook et al. (supra).

DNA may be stably incorporated into cells or may be transiently expressed using methods known in the art (see Sambrook et al., supra). Stably transfected mammalian cells may be prepared by transfecting cells with an expression vector having a selectable marker gene, and growing the transfected cells under conditions selective for cells expressing the marker gene. To prepare transient transfectants, mammalian cells are transfected with a reporter gene of interest, to monitor transfection efficiency. In one embodiment, the bait vector is introduced via infection using a viral vector such as adenoviral vectors, AAV vectors, retroviral vectors or lentiviral vectors.

Vectors of the invention can be present extrachromosomally or integrated into the host genome, and used to produce recombinant cells or organisms such as transgenic animals.

Plants and Plant Tissue

The method of the invention can be used to isolate DNA from any plant including but not limited Acer campestre, Aesculus hippocastanum, Alium ampeloprassum, Alium fistulosum, Alium porrum (seed), Alnus sp, Anethum graveolens, Anthericum liliago, Arabidopsis thaliana, Aristolochia macrophylla, Asparagus ojficinalis, Asplenim scolopendrium, Astragalus gummifer, Atropa belladona, Begonia sp., Beta vulgaris, Betula sp., Bletilla striata, Bombax sp., Brassica oleracea, Brunnera macrophylla, Buxus seinpervirens, Camellia sinensis, Caprinus sp., Caragana sophoriflora, Cardamine heptaphylla, Carex morrowii, Centaura macrocephala, Cercidiphylum japonicum, Chamaedorea microspadix, Clematis sp., Coffea arabica, Colchicum speciosum, Crocus albiflorus, Cyclamen purpurascens, Cymbidium pendulum, Danae recemosa, Daphne ponica, Dendrobium moschatrum, Dietes bicolor, Dipterracanthus devosianus, Epimedium alpinum, Eranthis hyemalis, Eryngium planum, Euonymus bungeana, Euphorbia leuconeura, Euphorbia rigida, Fragaria sp., Frenaria aurea, Fumaria capreolata, Gadiodus palustris, Geranium sp., Gloxinia sp., Glycine max (seed), Gossypium sp., Hedere helix, Helleborus dumentorum, Helleborus odoratus, Hibiscus magnifica, Humulus lupulus, Hycintus orientalis, Hypoestes sp., Ilex aquifolium, Impatiens sodenii, Inula ensifolia, Lactuca sativa (seed), Lathyrus vernus, Lilium henryi, Lilium pumilum, Liriope spicata, Lonicera caerulea, Lupinus sp., Lycopersicon esculentum, Mentha piperita, Narcissus pseudonarcissus, Nicotiana tabacum, Nymphea sp., Oreopanax sp., Oryza sativa, Paeonia belladona, Paeonia suffruticosa, Palisota manni, Papaya sp., Peperomia sp., Petasites albus, Phlomis fructica, Piper sp., Polygonum chinensis, Polygonum multiflorum, Primulapubescens, Primula vulgaris, Psychotria guadeloupensis, Rheum palntatum, Ribes petraeum, Rohdea japonica, Saintpaulia magungensis, Salvia officinalis, Saponaria officinale, Scilla bifolia, Setaria italica, Siningia sp., Sinningia magnifica, Sison amomum, Skimmia sp., Solanum tuberosum, Sorbus aria, Stachyfarpeta sp., Tilia sp., Tricantha affiifera, Triticum aestivum (seed), Triticum spelta, Triticum turgidum, Tulipa sp., Uniola latifolia, Urtica dioica, Vanhoutea sp., Veratrum album, Viburnum carlesii, Vitis vinifera, Weigeliafloribunda, Weigeliaprecox, Zea mais (leaf and seed).

The methods of the invention can be used to isolate DNA from any part of a plant, including but not limited to leaf, seed (including the cotyledon, embryo axis, root and stem), stem, flower, fruit, receptacle, floral tube, embryo, cotyledon, epidermis, phloem, xylem, parenchyma, endosperm and perisperm. DNA can be isolated from fresh plant material, from frozen leaves or from dried leaves, for example silica-dried leaves or herbarium specimens.

DNA Isolation from Plant Tissue.

The invention contemplates a method of isolating DNA from plant tissue using the mixture of cell wall degrading enzymes described herein.

In one embodiment, DNA is extracted from 5 mm leaf disks cut with a standard paper punch. Microtitration plates with 96 flat bottom wells are filled with 50 μl of digestion buffer and 3-5 μl of the enzymatic cocktail. One ml of a digestion buffer consists of 300 μl of 0.5 M EDTA, pH 8.0, 350 μl of 0.3 M sodium acetate adjusted to pH 4.6 with acetic acid, 10 μl of Triton X100, 10 μl of 25% polyethylene glycol (PEG) 8000 (Sigma P-5413) and 330 μl of water, and has a final pH of 5 to 6. The leaf disks are introduced in the wells and the plate is incubated from 1 to 16 hours, depending on the species, at 50° C. with agitation (250 rpm). After digestion of the leaf disks, the tissue is easily disrupted with a large hole pipette tip and 30 μl of the liquid containing the disrupted parenchymatic leaf tissue is collected in a microtube.

DNA is isolated by the addition of a solid substrate, for example, beads, either paramagnetic or silica based. Methods of immobilizing a nucleic acid sequence on a solid support are known in the art and are described in Ausubel FM et al. Current Protocols in Molecular Biology, John Wiley and Sons, Inc. and in protocols provided by the manufacturers, e.g., for membranes: Pall Corporation, Schleicher & Schuell, for magnetic beads: Promega, Dynal, Qiagen, for culture plates: Costar, Nalgenunc, and for other supports useful according to the invention, CPG, Inc. A solid support useful according to the invention includes but is not limited to silica based matrices, membrane based matrices and beads comprising surfaces including, but not limited to styrene, latex or silica based materials and other polymers. Paramagnetic beads are also useful according to the invention. Solid supports can be obtained from the above manufacturers and other known manufacturers.

In one embodiment, 200 μl of Dynabeads DNA DIRECT (DYNAL) are added following digestion, described above, and incubated 10 min. at 65° C. for complete cell lysis and release of DNA. DNA isolation is performed following the manufacturer's instructions and eluted in 40 μl of an elution buffer (recommended by the manufacturer). Ten μl of DNA is loaded on an ethidium bromide agarose gel to evaluate its quality and quantity.

In one embodiment, 150 μl of Lysis Buffer A, 72 μl of Lysis Buffer B and 5 μl of MagneSil of the Wizard® Magnetic 96 DNA Plant System (PROMEGA) are added following digestion, described above, and incubated 10 min. at room temperature for complete cell lysis and release of DNA. DNA isolation is performed following the manufacturer instructions and eluted in 40 μl of an elution buffer (recommended by the manufacturer). Ten μl of DNA is loaded on an ethidium bromide agarose gel to evaluate its quality and quantity.

The methods described herein using these compounds enable efficient DNA isolation without grinding of the tissues, without centrifugation and without deproteinization with phenol.

Preferably, DNA isolation is performed in the presence of a DNA preserving agent (EDTA), PEG, and non-ionic detergent (Triton-X-100).

The DNA isolated from plant tissues using the methods of the invention can be used for all molecular biology applications known in the art, including but not limited to PCR, cloning, sequencing and mutagenesis, AFLP, RAPD, RFLP, SSR, sequencing, Southern or partial mini-genomic libraries.

Kits

The invention herein also contemplates a kit format which comprises a package unit having one or more containers of the subject mixture of cell wall degrading enzymes and in some embodiments including containers of various reagents including a digestion buffer of the invention. In one embodiment, the digestion buffer comprises at least one of DNA preserving agent (for example EDTA), PEG and a non-ionic detergent (for example Triton-X-100). The kit may also contain one or more of the following items: additional buffers, instructions, and controls. Kits may include containers of reagents mixed together in suitable proportions for performing the methods in accordance with the invention. In one embodiment, a kit of the invention further comprises a solid support, as defined herein. Reagent containers preferably contain reagents in unit quantities that obviate measuring steps when performing the subject methods. Kits including packaging means, for example microfuge tubes, plates, 6, 12, 96 well etc . . . or membranes spotted with an aliquot of the mixture of cell wall degrading enzymes of the invention.

Automation

The method of the invention can be automated by using commercially available robots.

### EXAMPLES

The invention is illustrated by the following nonlimiting examples wherein the following materials and methods are employed. The entire disclosure of each of the literature references cited herein are incorporated by reference herein.

### Example 1

Purification of a Mixture of Cell Wall Degrading Enzymes from a Microorganism

A mixture of cell wall degrading enzymes is purified from the mutant fungal strain of Trichoderma longibrachiatum (TW-1 mutant) according to the following method.

Trichoderma longibrachiatum (TW-1 mutant) is cultivated in a fermenter on fermentation media with selected media components (media components—wheat bran+corn step solid+pre-hydrolised starch+lactose). At the end of the fermentation (144 hours) fungal cell biomass is removed by filtration (or by centrifugation, or by any other types of separation known in the art), and the cultural filtrate is concentrated by ultra-filtration. The resulting ultrafiltrate concentrate is a mixture of cell wall degrading enzymes that is used directly for the isolation of DNA from plant tissue.

### Example 2

Microscopic Analysis of Plant Leaves Incubated with a Mixture of Cell Wall Degrading Enzymes from a Microorganism

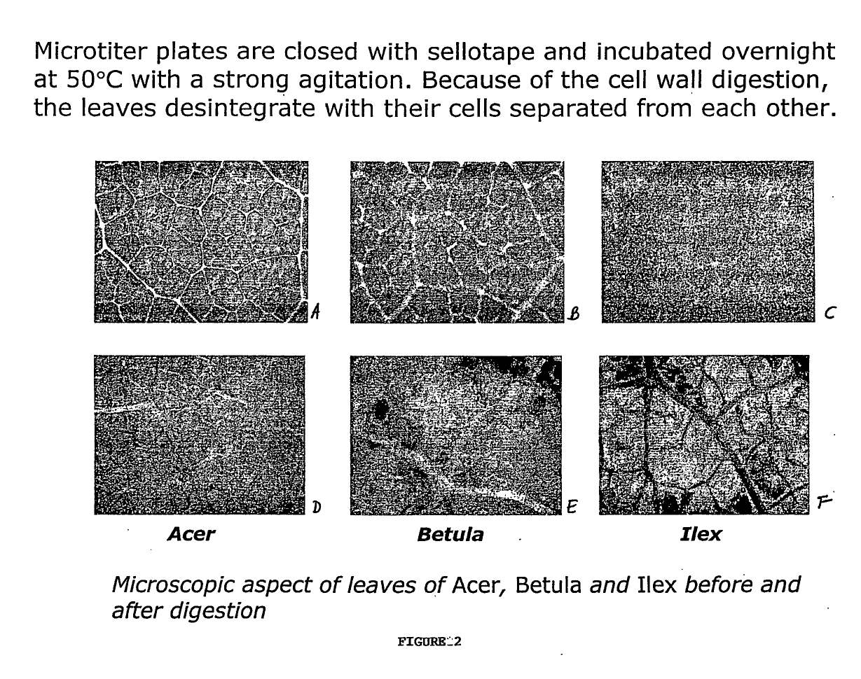

FIG. 2 shows the microscopic aspect of the leaves of three species (Acer sp., Betula sp. and Ilex aquifolium) before and after an overnight 50° C. incubation with a mixture of cell wall degrading enzymes from a microorganism. After incubation, the parenchymatic tissue is disrupted but the vascular vessels are not. This network of undigested vascular vessels often keeps the digested parenchymatic tissue in place. In order to visualize the digestion and to be able to collect the digested parenchymatic tissue it is necessary to lightly stir the leaf disk with a wide-bore pipette tip. The incubated enzymatic solution contains most of the digested parenchyrnatic tissue mainly composed in dissociated cells. Because of the high concentration of EDTA in the digestion buffer, the DNA is protected from intra- and inter-cellular DNAses.

### Example 3

Isolation of DNA from Plant Leaves Using Promega Paramagnetic Beads

DNA is isolated from plant leaf disks by incubation with a mixture of cell wall degrading enzymes from a microorganism.

In this example leaf disks of 48 randomly selected different plant species were treated with the mixture of cell wall degrading enzymes of the invention (3 μl). After the disruption of the digested parenchymatic tissue with a wide-bore pipette tip, 30 μl of liquid was transferred to a microtitration plate and the DNA was isolated with 150 μl of Lysis Buffer A, 72 μl of Lysis Buffer B and 5 μl of MagneSil of the Wizard® Magnetic 96 DNA Plant System kit (PROMEGA) using the MagnaBlot 96 Magnetic Separation Device (PROMEGA), following the manufacturer's instructions. Finally the DNA was eluted in 40 μl of an appropriate elution buffer (for example 10 mM Tris-HCl, pH 8.0 or water). Ten μl of this DNA was loaded on an ethidium-bromide agarose gel. In 39 out of the 48 purification reactions, DNA is visible on an ethidium bromide agarose gel (data not shown).

### Example 4

Isolation of DNA from Plant Leaves Using Dynal Paramagnetic Beads

DNA is isolated from Ilex Perado leaves by incubation with a mixture of cell wall degrading enzymes from a microorganism.

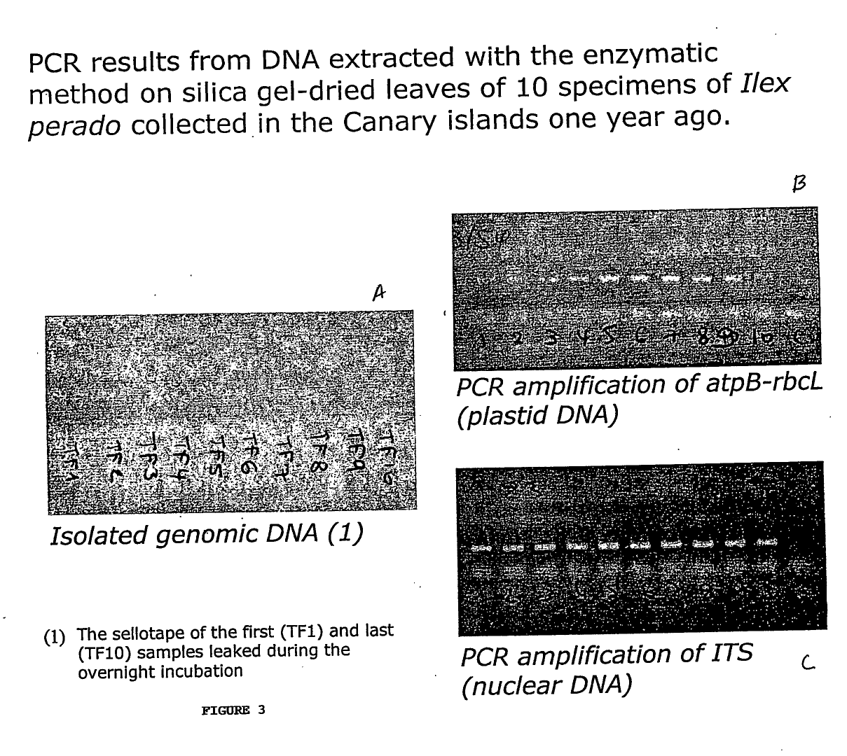

In this example leaf disks of 10 different Ilex perado plants were treated with the mixture of cell wall degrading enzymes of the invention (3-5 μl). After the disruption of the digested parenchymatic tissue with a wide-bore pipette tip, 30 μl of liquid was transferred to a new micro-tube and the DNA was isolated using 200 μl of Dynabeads DNA DIRECT (magnetic beads from DYNAL), following the manufacturer's instructions. Finally the DNA was eluted in 40 μl of an appropriate elution buffer (for example 10 mM Tris-HCl, pH 8.0, or H₂O). Ten μl of this DNA was loaded on an ethidium-bromide agarose gel. One μl of this DNA was used to amplify nuclear (ITS) and chloroplast (atpB-rbcL spacer) sequences (FIG. 3).

### Example 5

Isolation of DNA from Plant Leaves of a Variety of Species

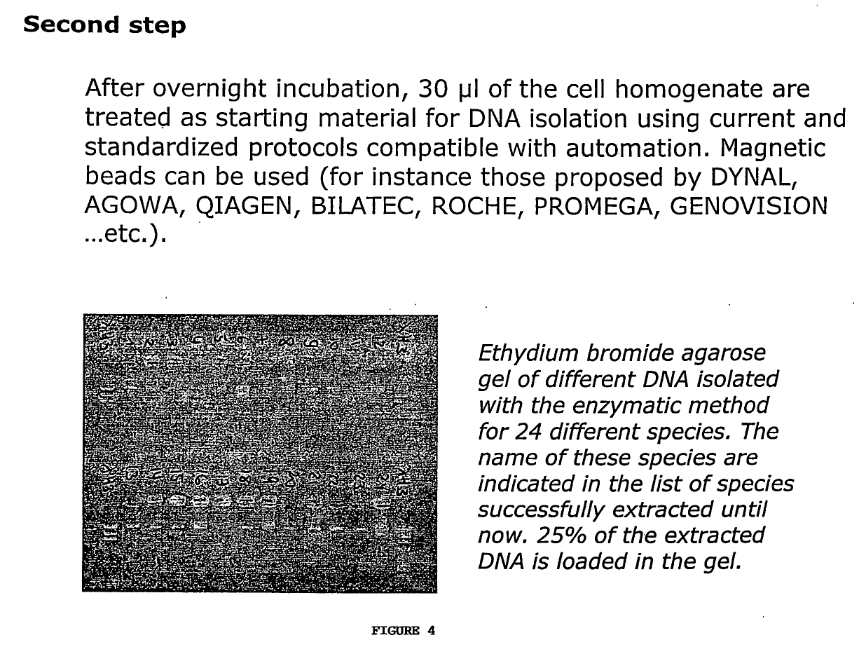

In this example total DNA was isolated from a leaf disk isolated from 24 different species. FIG. 4 shows an ethidium bromide agarose gel of the DNA isolated with the described enzymatic method from 24 different species. 25% of the extracted DNA (10 μl) was loaded in the gel. From 1 to 24 are the following species: Phlomis fructica, Humulus lupulus, Veratrum album, Scilla bifolia, Astragalus gummifer, Vitis vinifera, Centaura macrocephala. Narcissus pseudonarcissus, Alium ampeloprassum, Salvia officinalis, Viburnum carlesii, Colchicum speciosum, Triticum turgidum, Polygonum chinensis, Lathyrus vernus, Tilia sp., Caraganax sophoriflora, Urtica dioica, Lilium henryi, Polygonum multiflorus, Geranium sp., Lupinus sp., Crocus albiflorus, Helleborus dumentorum. Left and right: 2.5 μg of Hind III digested Lambda DNA as size marker. The comparison of the intensity of the plant DNA bands with the intensity of the 23 kbp Lambda fragment (representing 1.2 μg) allows an estimation of the amount of total DNA isolated by this method from a leaf disk.

### Example 6

Isolation of DNA from Plant Seeds

The enzyme mixture of the invention was used to isolate DNA from plant seeds.

Dry seeds of wheat, corn, soy, lettuce and leek have been tested by the enzymatic method. In order to allow the penetration of enzymes, small seeds (lettuce and leek) were cut in two pieces, large seeds were fragmented to a size of 2-3 mm and the incubation was done in 50 μl of digestion buffer and 3 μl of the mixture of cell wall degrading enzymes of the invention Large amounts of high quality DNA were isolated (data not shown).

### Example 7

Preparation of a Recombinant Cell Wall Degrading Enzyme

A cell wall degrading enzyme of the mixture of the invention is prepared using recombinant methods well known in the art. A nucleic acid encoding a cell wall degrading enyzme, for example as described in the section entitled “Preparation of a mixture of cell wall degrading enzymes via recombinant methods” is cloned into an appropriate expression vector using conventional methods well known in the art. The vector is expressed by introduction of the vector into an appropriate host cell. A host cell expressing the vector is identified by methods, known in the art, for example, DNA-DNA hybridization. A host cell that is transformed with the vector of interest is cultured under conditions suitable for expression and recovery of the cell wall degrading enzyme of interest.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

### OTHER EMBODIMENTS

Other embodiments will be evident to those of skill in the art. It should be understood that the foregoing detailed description is provided for clarity only and is merely exemplary. The spirit and scope of the present invention are not limited to the above examples, but are encompassed by the following claims.

1. A composition comprising a mixture of cell wall degrading enzymes.

2. The composition of claim 1, wherein said enzymes of said composition are produced recombinantly.

3. The composition of claim 1, wherein said mixture is isolated from a microorganism.

4. The composition of claim 3, wherein said microorganism is a fungus.

5. The composition of claim 4, wherein said fungus is selected from the group consisting of: Trichoderma, Pencillium and Aspergillus.

6. The composition of claim 4 wherein said fungus is the TW-1 mutant strain of Trichoderma longibrachiatum.

7. The composition of claim 1, wherein said enzymes comprise carbohydrases.

8. The composition of claim 1, wherein said mixture comprises cellulases, β-glucanases, mannanases, xyloglucanases, pectinases,glycosidases and xylanases.

9. The composition of claim 1, wherein said mixture comprises at least one of the enzymes selected from the group consisting of cellulases, β-glucanases, mannanases, xyloglucanases, pectinases, glycosidases and xylanases.

10. The composition of claim 1 further comprising a digestion buffer comprising a DNA preserving agent.

11. The composition of claim 10, wherein said DNA preserving agent is EDTA.

12. The composition of claim 10, wherein said digestion buffer further comprises at least one of a non-ionic detergent and PEG.

13. The composition of claim 12 wherein said detergent is Triton-X-100.

14. The composition of claim 10, wherein said digestion buffer has a pH of 5.0.

15. A method for isolating DNA from plant tissue comprising:
combining a sample of plant tissue with a mixture of cell wall degrading enzymes, and
incubating said plant tissue and said mixture of cell wall degrading enzymes.

16. The method of claim 15, wherein said enzymes of said mixture are produced recombinantly.

17. The method of claim 15, wherein said mixture is isolated from a microorganism.

18. The method of claim 15, wherein said microorganism is a fungus.

19. The method of claim 18, wherein said fungus is selected from the group consisting of: Trichoderma, Pencillium and Aspergillus.

20. The method of claim 18 wherein said fungus is the TW-1 mutant strain of Trichoderma longibrachiatum.

21. The method of claim 15, wherein said enzymes comprise carbohydrases.

22. The method of claim 15, wherein said mixture comprises cellulases, β-glucanases, mannanases, xyloglucanases, pectinases, glycosidases and xylanases.

23. The method of claim 15, wherein said mixture comprises at least one of cellulases, β-glucanases, mannanases, xyloglucanases, pectinases, glycosidases and xylanases.

24. The method of claim 15, wherein said incubation is performed in the presence of a digestion buffer comprising a DNA preserving agent.

25. The method of claim 24, wherein said DNA preserving agent is EDTA.

26. The method of claim 24 wherein said digestion buffer further comprises at least one of a non-ionic detergent and PEG.

27. The method of claim 26, wherein said detergent is Triton-X-100.

28. The method of claim 24, wherein said buffer has a pH of 5.0.

29. The method of claim 15, wherein said incubation is performed at 50° C.

30. The method of claim 15, wherein said combination of said mixture of cell wall degrading enzymes and said sample are agitated at 250 rpm for 1-16 hours.

31. The method of claim 15, further comprising the steps of adding a DNA-binding solid support and binding said DNA to said solid support after said incubation step.

32. The method of claim 15, wherein said method is automated.

33. A kit for isolating DNA from plant tissue comprising a mixture of cell wall degrading enzymes and packaging means thereof.

34. The kit of claim 33, wherein said enzymes of said mixture are prepared recombinantly.

35. The kit of claim 34, wherein said mixture is isolated from a microorganism.

36. The kit of claim 35, wherein said microorganism is a fungus.

37. The kit of claim 36, wherein said fungus is selected from the group consisting of: Trichoderma, Pencillium and Aspergillus.

38. The kit of claim 36 wherein said fingus is the TW-1 mutant strain of Trichoderma longibrachiatum.

39. The kit of claim 33, wherein said enzymes comprise carbohydrases.

40. The kit of claim 33, wherein said mixture comprises cellulases, β-glucanases, mannanases, xyloglucanases, pectinases, glycosidases and xylanases.

41. The kit of claim 33, wherein said mixture comprises at least one of cellulases, β-glucanases, mannanases, xyloglucanases, pectinases, glycosidases and xylanases.

42. The kit of claim 33, further comprising a digestion buffer comprising a DNA preserving agent.

43. The kit of claim 42, wherein said DNA preserving agent is EDTA.

44. The kit of claim 42 wherein said digestion buffer further comprises at least one of a non-ionic detergent and PEG.

45. The kit of claim 44, wherein said detergent is Triton-X-100.

46. The kit of claim 42, wherein said digestion buffer has a pH of 5.0.

47. The kit of claim 33, further comprising a DNA-binding solid support.
